(19) United States
(12) Patent Application Publication
(10) Pub. No.: US 2025/0102506 A1
(43) Pub. Date: Mar. 27, 2025

(54) COMPOSITION FOR DIAGNOSING CANCER

(71) Applicant: BERTIS INC, Gwacheon-si Gyeonggi-do (KR)

(72) Inventors: Sungsoo KIM, Yongin-si, Gyeonggi-do (KR); Youngmin PARK, Suwon-si Gyeonggi-do (KR); Hyeonseok SHIN, Yongin-si, Gyeonggi-do (KR); Seungman HAN, Seoul (KR); Dongyoung NOH, Seoul (KR)

(73) Assignee: BERTIS INC, Gwacheon-si Gyeonggi-do (KR)

(21) Appl. No.: 18/722,482

(22) PCT Filed: Mar. 30, 2023
(86) PCT No.: PCT/KR2023/004274

§ 371 Date: Dec. 12, 2024

(30) Foreign Application Priority Data
Mar. 31, 2022 (KR) 10-2022-0040225

(51) Int. Cl. G01N 33/574; G01N 33/573; G01N 33/68; G16B 20/00

(52) U.S. Cl. CPC G01N 33/57415; G01N 33/573; G01N 33/6848; G16B 20/00; G01N 2333/95; G01N 2333/976

(57) ABSTRACT

The present invention relates to a method of diagnosing cancer using proteins (or fragments thereof), which are specifically expressed in cancer, or genes encoding the proteins. According to the present invention, specific proteins and fragments thereof useful for cancer diagnosis are discovered among blood proteins, and they may be used as diagnostic biomarkers to diagnose in particular breast cancer simply and accurately at an early stage, thereby significantly lowering the mortality rate of patients with related diseases.

### TECHNICAL FIELD

The present invention relates to a method of diagnosing breast cancer using mass spectrometry.

### BACKGROUND ART

Breast cancer is a cancer that has become the highest incidence of cancer in women worldwide, and is also a fatal cancer that is the first leading cause of death among women. Conventional methods for screening breast cancer rely on imaging methods. In particular, mammography, an imaging method for diagnosis of breast cancer, has disadvantages in that there are concerns about risks of excessive radiation exposure, there are limitations in that the diagnostic accuracy for dense breasts is low, and body exposure and compression cause discomfort and pain to the patient. In addition, breast ultrasound examination is an expensive examination method that limits user accessibility, and the determination of the results may vary depending on the tester's skill level or the degree of equipment deterioration. Due to these disadvantages of the conventional art, there is a high demand for an easy, simple, and cost-effective testing method in the field of early diagnosis of breast cancer, and a blood test is considered one of the most suitable methods.

The existing blood tests for breast cancer include the CA15-3 immunoassay, which was developed in the 1980s and approved by the US FDA in 1997. However, the CA15-3 immunoassay has a low diagnostic accuracy of 10 to 20% for early-stage breast cancer, and thus is used for monitoring patients under treatment rather than for early diagnosis.

Meanwhile, in recent clinical practice, there is a recognition that it is difficult to make an accurate diagnosis with a single marker test, and multiple markers are emerging as an alternative to solve this problem. Under this recognition, mass spectrometry is considered as a method suitable for using multiple markers in that it can measure a large number of markers simultaneously and does not use antibodies. However, although studies on the discovery of thousands of biomarkers through mass spectrometry have continued, mass spectrometry is still rarely applied clinically due to issues of high price, low reproducibility, and long analysis time.

In order for mass spectrometry to be well applied in clinical practice, price competitiveness and reproducibility have to be ensured, and analysis time should also be significantly reduced. In particular, in order to ensure both reproducibility and economic efficiency in the blood protein pretreatment process, it is necessary to preprocess blood without depletion of highly abundant proteins, and it is also essential to drastically reduce the analysis time from the existing 1 to 2 hours to 10 to 20 minutes. Depletion of highly abundant proteins that are unnecessary for analysis can increase the number of protein identifications (profiling), but some analytes are also depleted in the process of depleting highly abundant proteins, and it is difficult to guarantee reproducibility because the degree of protein depletion varies between samples or between columns. In addition, due to the nature of having to be subjected to multiple steps, the handling error rate increases and the analysis time becomes longer, which causes a significant increase in cost.

Accordingly, the present inventors have sought to discover breast cancer-screening biomarkers which may be analyzed in blood (serum and plasma) without depletion of highly abundant proteins such as albumin, immunoglobulin, and transferrin, may drastically reduce the analysis time to less than 10 minutes, and ultimately are economically feasible and reproducible enough for clinical use.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### DISCLOSURE

### Technical Problem

In order to solve excessive radiation exposure and the low diagnostic accuracy for dense breasts, which are the disadvantages of mammography, a conventional diagnostic method for breast cancer, the present inventors have made extensive research efforts to develop a method that enables simple and rapid analysis by analyzing blood without depletion of highly abundant proteins. As a result, the present inventors have discovered biomarkers that are quantifiable and reproducible enough for use in clinical settings, and have found that breast cancer may be diagnosed quickly and with high reliability using the biomarkers, thereby completing the present invention.

Therefore, an object of the present invention is to provide a composition for diagnosing cancer.

Another object of the present invention is to provide a kit for diagnosing cancer.

Still another object of the present invention is to provide a method of providing information for diagnosing cancer.

Yet another object of the present invention is to provide a method for screening a composition for preventing or treating cancer.

Other objects and advantages of the present invention will be more apparent from the following detailed description, the appended claims and the accompanying drawings.

### Technical Solution

According to one aspect of the present invention, the present invention provides a composition for diagnosing cancer, comprising an agent for measuring the expression level of at least one polypeptide selected from the group consisting of APOC1 (apolipoprotein C1), CHL1 (neural cell adhesion molecule L1-like), MMP9 (matrix metalloproteinase-9), PRDX6 (peroxiredoxin-6), PRG4 (proteoglycan 4), PPBP (platelet basic protein), FN1 (fibronectin), VWF (von Willebrand factor), and CLU (clusterin), or a fragment thereof, or a gene encoding the polypeptide or fragment thereof.

In order to solve excessive radiation exposure and the low diagnostic accuracy for dense breasts, which are the disadvantages of mammography, a conventional diagnostic method for breast cancer, the present inventors have made extensive research efforts to develop a method that enables simple and rapid analysis by analyzing blood without depletion of highly abundant proteins. As a result, the present inventors have discovered biomarkers that are quantifiable and reproducible enough for use in clinical settings, and have found that breast cancer may be diagnosed quickly and with high reliability using the biomarkers, thereby completing the present invention.

In the present invention, the agent for measuring the expression level of the polypeptide may be an agent for measuring the expression level of apolipoprotein C1 (APOC1). APOC1 is a member of the apolipoprotein C family, and may be encoded by the APOC1 gene in humans. In addition, it is first expressed in the liver and may later be activated when monocytes differentiate into macrophages.

In the present invention, the agent for measuring the expression level of the polypeptide may be an agent for measuring the expression level of CHL1. CHL1 refers to “Close Homolog of L1”, may also be called neural cell adhesion molecule L1 like protein, and may be encoded by the CHL1 gene in humans.

In the present invention, the agent for measuring the expression level of the polypeptide may be an agent for measuring the expression level of matrix metalloproteinase-9 (MMP9). MMP-9 is also known as 92-kDa type IV collagenase, 92-kDa gelatinase, or gelatinase B (GELB). MMP-9 is a member of the zinc-metalloproteinases family and is known to be involved in degrading the extracellular matrix. It may be encoded by the MMP9 gene in humans.

In the present invention, the agent for measuring the expression level of the polypeptide may be an agent for measuring the expression level of peroxiredoxin-6 (PRDX6). PRDX6 may be a member of the ferredoxin family, which is an antioxidant enzyme, and may be encoded by the PRDX6 gene in humans.

In the present invention, the agent for measuring the expression level of the polypeptide may be an agent for measuring the expression level of proteoglycan 4 (PRG4). PRDG4 is also called lubricin, and may be encoded by the Prg4 gene in humans.

In the present invention, the agent for measuring the expression level of the polypeptide may be an agent for measuring the expression level of pro-platelet basic protein (PPBP). PPBP is also called a chemokine (C-X-C motif) ligand (CXCL7), and may be encoded by the PPBP gene in humans.

In the present invention, the agent for measuring the expression level of the polypeptide may be an agent for measuring the expression level of fibronectin (FN1). FN1 is a high molecular weight glycoprotein that may be attached to integrin, a membrane receptor protein of the extracellular matrix, and may bind to other extracellular matrix proteins such as collagen, fibrin, and heparan sulfate proteoglycans. In addition, it may be encoded by the FN1 gene in humans.

In the present invention, the agent for measuring the expression level of the polypeptide may be an agent for measuring the expression level of von Willebrand factor (VWF). VWF is an adhesion factor produced by vascular endothelial cells or the megakaryocytes of bone marrow. It may act as an adhesive when platelets bind to subendothelial tissue, or as a co-factor for coagulation factor VIII, and function to induce stabilization by binding to factor VIII in the blood. In addition, it may be encoded by the VWF gene in humans.

In the present invention, the agent for measuring the expression level of the polypeptide may be an agent for measuring the expression level of clusterin (CLU). The clusterin protein is a disulfide-linked heterodimeric protein with a molecular weight of 75 to 80 kDa, and is known to be involved in cell debris removal and cell death. In addition, it may be encoded by the CL U gene in humans.

In the present invention, the “cancer” as a disease to be diagnosed refers to or describes a physiological condition in mammals that is typically characterized by unregulated cell growth. In the present invention, the cancer to be diagnosed may be specifically breast cancer, ovarian cancer, colorectal cancer, gastric cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, cancer of ureter, renal cell carcinoma, renal pelvic carcinoma, central nervous system (CNS) tumor, primary CNS lymphoma, spinal cord tumor, brainstem glioma, or pituitary adenoma. More specifically, the cancer may be breast cancer.

In the present invention, the “diagnosis” or “diagnosing” includes: determining the susceptibility of a subject to a specific disease or disorder; determining whether or not a subject currently has a particular disease or disorder; determining the prognosis of a subject with a specific disease or disorder (e.g., identification of pre-metastatic or metastatic cancer conditions, determination of cancer stages, or determination of responsiveness of cancer to therapy); or therametrics (e.g., monitoring states of a subject to provide information about treatment effects). With regard to the purposes of the present invention, the diagnosis or diagnosing refers to determining whether or not the above-described cancer has developed or the likelihood (risk) of onset of the cancer.

In the present invention, the agent for measuring the expression level of the polypeptide is not particularly limited, but may comprise, for example, at least one selected from the group consisting of an antibody, an oligopeptide, a ligand, a peptide nucleic acid (PNA), and an aptamer, which bind specifically to the polypeptide.

In the present invention, the “antibody” refers to a substance that binds specifically to an antigen, causing an antigen-antibody reaction. With regard to the purposes of the present invention, the antibody refers to antibodies that bind specifically to the polypeptides mentioned in the present invention.

The antibodies of the present invention include all polyclonal antibodies, monoclonal antibodies, and recombinant antibodies. The antibody may be easily produced using techniques well known in the art. For example, the polyclonal antibody may be produced by a method well known in the art, which comprises a process of injecting the protein antigen into an animal, collecting blood from the animal, and isolating serum containing the antibody. This polyclonal antibody may be produced from any animal species such as goats, rabbits, sheep, monkeys, horses, pigs, cattle, or dogs. In addition, the monoclonal antibody may be produced using a hybridoma method (see Kohler and Milstein (1976) European Journal of Immunology 6:511-519) well known in the art, or phage antibody library technology (see Clackson et al, Nature, 352:624-628, 1991; Marks et al, J. Mol. Biol., 222:58, 1-597, 1991). The antibody produced by the above method may be isolated and purified using methods such as gel electrophoresis, dialysis, salt precipitation, ion exchange chromatography, and affinity chromatography. In addition, the antibodies of the present invention include functional fragments of antibody molecules as well as complete forms having two full-length light chains and two full-length heavy chains. The “functional fragments of antibody molecules” refers to fragments retaining at least an antigen-binding function, and examples of the functional fragments include Fab, F(ab′), F(ab′)2, and Fv.

In the present invention, the “peptide nucleic acid (PNA)” refers to an artificially synthesized polymer similar to DNA or RNA, and was first introduced by professors Nielsen, Egholm, Berg and Buchardt (at the University of Copenhagen, Denmark) in 1991. DNA has a phosphate-ribose backbone, whereas PNA has a backbone composed of repeating units of N-(2-aminoethyl)-glycine linked by peptide bonds. Thanks to this structure, PNA has a significantly increased binding affinity for DNA or RNA and a significantly increased stability, and thus is used in molecular biology, diagnostic analysis, and antisense therapy. PNA is disclosed in detail in Nielsen PE, Egholm M, Berg RH, Buchardt O (December 1991). “Sequence-selective recognition of DNA by strand displacement with a thymine-substituted polyamide”. Science 254(5037): 1497-1500.

In the present invention, the “aptamer” is an oligonucleic acid or peptide molecule, and general contents of the aptamer are disclosed in detail in Bock LC et al., Nature 355(6360):5646(1992); Hoppe-Seyler F, Butz K “Peptide aptamers: powerful new tools for molecular medicine”. J Mol Med. 78(8):42630(2000); Cohen BA, Colas P, Brent R. “An artificial cell-cycle inhibitor isolated from a combinatorial library”. Proc Natl Acad Sci USA. 95(24): 142727(1998).

In the present invention, the agent for measuring the expression level of the gene encoding the polypeptide may comprise at least one selected from the group consisting of a primer, a probe, and an antisense oligonucleotide, which bind specifically to the gene.

In the present invention, the “primer” is a fragment that recognizes a target gene sequence, and includes a pair of forward and reverse primers. Preferably, the primer is a primer pair that provides analysis results with specificity and sensitivity. Because the nucleotide sequence of the primer does not match a non-targeted sequence in a sample, the primer can show high specificity when it amplifies only a target gene sequence containing a complementary primer binding site without causing non-specific amplification.

In the present invention, the “probe” refers to a substance which is capable of binding specifically to the target substance to be detected in a sample and may specifically identify the presence of the target substance in the sample through the binding. The kind of the probe is not specifically limited, as long as it is a substance that is generally used in the art. Preferably, the probe may be peptide nucleic acid (PNA), locked nucleic acid (LNA), a peptide, a polypeptide, a protein, RNA or DNA. Most preferably, the probe is PNA. More specifically, the probe may be a biomaterial derived from an organism, an analogue thereof, or a material produced ex vivo, and examples thereof include enzymes, proteins, antibodies, microorganisms, animal/plant cells and organs, neural cells, DNA, and RNA. Examples of the DNA include cDNA, genomic DNA, and oligonucleotides, examples of the RNA include genomic RNA, mRNA, and oligonucleotides, and examples of the protein include antibodies, antigens, enzymes, and peptides.

In the present invention, the “locked nucleic acid (LNA)” refers to a nucleic acid analog containing a 2′-O or 4′-C methylene bridge [J Weiler, J Hunziker and J Hall Gene Therapy (2006) 13, 496.502]. LNA nucleosides include common nucleic acid bases of DNA and RNA, and can form base pairs according to the Watson-Crick base pairing rule. However, due to ‘locking’ of the molecule attributable to the methylene bridge, the LNA fails to form an ideal shape in the Watson-Crick bond. When the LNA is incorporated in a DNA or RNA oligonucleotide, it can more rapidly pair with a complementary nucleotide chain, thus increasing the stability of the double strand. In the present invention, the “antisense” refers to an oligomer having a sequence of nucleotide bases and a subunit-to-subunit backbone that allows the antisense oligomer to hybridize to a target sequence in an RNA by Watson-Crick base pairing, to form an RNA:oligomer heteroduplex within the target sequence, typically with an mRNA. The oligomer may have exact sequence complementarity to the target sequence or near complementarity.

Since information on the amino acid sequence of the polypeptide according to the present invention and on the nucleic acid sequence encoding the polypeptide is available from various public data sources, those skilled in the art may easily design a primer, a probe, or an antisense oligonucleotide, which bind specifically to the gene encoding the polypeptide, based on the information.

According to a specific embodiment of the present invention,
the fragment of the APOC1 polypeptide has the amino acid sequence of SEQ ID NO:1 (TPDVSSALDK);
the fragment of the CHL1 polypeptide has the amino acid sequence of SEQ ID NO:2 (VIAVNEVGR);
the fragment of the MMP9 polypeptide has the amino acid sequence of SEQ ID NO:3 (AVIDDAFAR);
the fragment of the PRDX6 polypeptide has the amino acid sequence of SEQ ID NO:4 (LSILYPATTGR);
the fragment of the PRG4 polypeptide has the amino acid sequence of SEQ ID NO:5 (AIGPSQTHTIR);
the fragment of the PPBP polypeptide has the amino acid sequence of SEQ ID NO:6 (TTSGIHPK);
the fragment of the FN1 polypeptide has the amino acid sequence of SEQ ID NO:7 (STTPDITGYR);
the fragment of the VWF polypeptide has the amino acid sequence of SEQ ID NO:8 (ILAGPAGDSNVVK); and
the fragment of the CLU polypeptide has the amino acid sequence of SEQ ID NO:9 (TLLSNLEEAK).

According to a specific embodiment of the present invention, the cancer that may be diagnosed using the composition for diagnosing according to the present invention is breast cancer.

According to a specific embodiment of the present invention, the agent for measuring the expression level of the polypeptide comprises at least one selected from the group consisting of an antibody, an oligopeptide, a ligand, a peptide nucleic acid (PNA), and an aptamer, which bind specifically to the polypeptide or a fragment thereof.

According to a specific embodiment of the present invention, the agent for measuring the expression level of a gene encoding the polypeptide or fragment thereof comprises at least one selected from the group consisting of a primer, a probe, and an antisense oligonucleotide, which bind specifically to the gene.

According to another aspect of the present invention, the present invention provides a diagnostic kit comprising the composition for diagnosing according to the present invention.

In the present invention, it is possible to diagnose the onset, likelihood of onset, responsiveness to therapy, prognosis, stage, likelihood of recurrence, etc. of cancer disease using the above diagnostic kit.

In the present invention, the cancer to be diagnosed has already been described in detail, and thus detailed description thereof will be omitted below to avoid excessive overlapping.

In the present invention, the kit may be, but is not limited to, an RT-PCR kit, a DNA chip kit, an ELISA kit, a protein chip kit, a rapid kit or a multiple-reaction monitoring (MRM) kit.

The cancer diagnostic kit of the present invention may further comprise one or more other component compositions, solutions or devices suitable for analysis methods.

For example, the cancer diagnostic kit of the present invention may further comprise essential elements necessary for performing reverse transcription polymerase reaction. The reverse transcription polymerase reaction kit comprises a pair of primers specific to a gene encoding a marker protein. Each primer is an oligonucleotide having a sequence specific to the nucleic acid sequence of the gene, and may have a length of about 7 bp to 50 bp, more preferably about 10 bp to 30 bp. In addition, the kit may comprise primers specific to the nucleic acid sequence of a control gene. In addition, the reverse transcription polymerase reaction kit may comprise a test tube or other suitable container, buffers (having various pHs and magnesium concentrations), deoxynucleotides (dNTPs), enzymes such as Taq-polymerase and reverse transcriptase, DNAse and RNAse inhibitors, DEPC-water, sterile water, and the like.

In addition, the diagnostic kit of the present invention may comprise essential elements necessary for performing DNA chip assay. The DNA chip kit may comprise a substrate to which a gene or a cDNA or oligonucleotide corresponding to a fragment thereof is attached, and reagents, agents, and enzymes for constructing a fluorescently labeled probe. In addition, the substrate may comprise a control gene or a cDNA or oligonucleotide corresponding to a fragment thereof.

In addition, the diagnostic kit of the present invention may comprise essential elements necessary for performing ELISA. The ELISA kit may comprise an antibody specific to the protein. The antibody has high specificity and affinity for the marker protein, with little cross-reactivity to other proteins, and may be a monoclonal antibody, a polyclonal antibody, or a recombinant antibody. Furthermore, the ELISA kit may comprise an antibody specific to a control protein. In addition, the ELISA kit may further comprise reagents capable of detecting the bound antibody, for example, a labeled secondary antibody, chromophores, an enzyme (e.g., conjugated with the antibody) and a substrate thereof, or other substances capable of binding to the antibody.

In the diagnostic kit of the present invention, as a fixture for antigen-antibody binding reaction, a well plate synthesized from a nitrocellulose membrane, a PVDF membrane, a polyvinyl resin or a polystyrene resin, or a glass slide made of glass may be used, without being limited thereto.

In addition, in the diagnostic kit of the present invention, a label for the secondary antibody is preferably a conventional chromogenic agent for color development, and examples of the label include, but are not limited to, fluoresceins such as HRP (horseradish peroxidase), alkaline phosphatase, colloid gold, FITC (poly L-lysine-fluorescein isothiocyanate), RITC (rhodamine-B-isothiocyanate), and dyes.

In addition, in the diagnostic kit of the present invention, a chromogenic substrate for inducing color development is preferably selected depending on the label for color development, and may be TMB (3,3′,5,5′-tetramethyl benzidine), ABTS [2,2′-azino-bis(3-ethylbenzothiazoline-6-sulfonic acid)], or OPD (o-phenylenediamine). At this time, the chromogenic substrate is more preferably provided as dissolved in buffer (0.1M NaOAc, pH 5.5). A chromogenic substrate such as TMB is degraded by HRP, used as a label for the secondary antibody conjugate, to form a chromogen, and the presence of the marker protein is detected by visually checking the degree of deposition of the chromogen.

The washing solution in the diagnostic kit of the present invention preferably comprises phosphate buffer, NaCl and Tween 20. More preferably, the washing solution is a buffer solution (PBST) consisting of 0.02 M phosphate buffer, 0.13 M NaCl, and 0.05% Tween 20. After the antigen-antibody binding reaction, the secondary antibody is allowed to react with the antigen-antibody complex, and then the resulting conjugate is washed 3 to 6 times with a suitable amount of the washing solution added to the fixture. As the reaction stop solution, a sulfuric acid solution (H₂SO₄) is preferably used.

According to a specific embodiment of the present invention, the diagnostic kit of the present invention is an RT-PCR kit, a DNA chip kit, an ELISA kit, a protein chip kit, a rapid kit or a multiple-reaction monitoring (MRM) kit.

According to another aspect of the present invention, the present invention provides a method of providing information for diagnosing cancer, comprising a step of measuring the expression level of at least one polypeptide selected from the group consisting of APOC1 (apolipoprotein C1), CHL1 (neural cell adhesion molecule L1-like), MMP9 (matrix metalloproteinase-9), PRDX6 (peroxiredoxin-6), PRG4 (proteoglycan 4), PPBP (platelet basic protein), FN1 (fibronectin), VWF (von Willebrand factor), and CLU (clusterin), or a fragment thereof, or a gene encoding the polypeptide or fragment thereof, in a biological sample isolated from a subject of interest.

In the present invention, the polypeptides whose expression levels are to be measured have already been described in detail, and thus detailed description thereof will be omitted below to avoid excessive overlapping.

In the present invention, the “subject of interest” refers to a subject in whom whether or not the cancer has developed is uncertain and who has a high likelihood of onset of the cancer.

In the present invention, the “biological sample” refers to any material, biological fluid, tissue or cells obtained or derived from the subject. For example, the biological sample may include whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid. Preferably, the biological sample may be a liquid biopsy (e.g., patient's tissue, cells, blood, serum, plasma, saliva, sputum or ascites, etc.) collected for histopathological examination by inserting a hollow needle or the like into an in vivo organ without incision of the skin of a patient having a high likelihood of onset of cancer.

The method of the present invention may comprise a step of measuring the expression levels of the polypeptides represented by SEQ ID NO:1 to 9 or genes encoding the same in the biological sample isolated as described above.

In the present invention, the step of measuring the expression level may be a step of measuring the expression level of at least one protein (polypeptide) selected from the group consisting of APOC1 (apolipoprotein C1), CHL1 (neural cell adhesion molecule L1-like), MMP9 (matrix metalloproteinase-9), PRDX6 (peroxiredoxin-6), PRG4 (proteoglycan 4), PPBP (platelet basic protein), FN1 (fibronectin), VWF (von Willebrand factor), and CLU (clusterin), or a gene encoding the protein.

In the present invention, an agent for measuring the expression level of the polypeptide is not particularly limited, but may comprise, for example, at least one selected from the group consisting of an antibody, an oligopeptide, a ligand, a peptide nucleic acid (PNA), and an aptamer, which bind specifically to the polypeptide.

In the present invention, methods for measurement or comparative analysis of the expression level of the polypeptide include, but are not limited to, protein chip assay, immunoassay, ligand binding assay, MALDI-TOF (Matrix Assisted Laser Desorption/Ionization Time of Flight Mass Spectrometry) assay, SELDI-TOF (Surface Enhanced Laser Desorption/Ionization Time of Flight Mass Spectrometry) assay, radioimmunoassay, radioimmunodiffusion, Ouchterlony immunodiffusion, rocket immunoelectrophoresis, immunohistochemical staining, complement fixation assay, two-dimensional electrophoresis assay, liquid chromatography-mass spectrometry (LC-MS), LC-MS/MS (liquid chromatography-mass spectrometry/mass spectrometry), Western blotting, and ELISA (enzyme-linked immunosorbent assay).

In the present invention, a method for measurement or comparative analysis of the expression level of the polypeptide may be performed by a multiple reaction monitoring (MRM) method.

In the present invention, the multiple-reaction monitoring method may be performed using mass-spectrometry, preferably triple-quadrupole mass spectrometry.

In the present invention, the multiple-reaction monitoring (MRM) method using mass-spectrometry is an analysis technique capable of monitoring a change in concentration of a specific analyte by selectively isolating, detecting and quantifying the specific analyte. MRM is a method that can quantitatively and accurately measure multiple substances such as trace amounts of biomarkers present in a biological sample. In MRM, mother ions among the ion fragments generated in an ionization source are selectively transmitted to a collision tube by a first mass filter Q1. Then, the mother ions arriving at the collision tube collide with an internal collision gas, are fragmented to generate daughter ions which are then sent to a second mass filter Q2, where only characteristic ions are transmitted to a detection unit. MRM is an analysis method with high selectivity and sensitivity that can detect only information on a component of interest. MRM is used for quantitative analysis of small molecules and is used to diagnose specific genetic diseases. The MRM method has advantages in that it is easy to simultaneously measure multiple peptides, and it is possible to confirm the relative concentration difference of protein diagnostic marker candidates between a normal person and a cancer patient without using an antibody. In addition, since the MRM analysis method has excellent sensitivity and selectivity, it has been introduced for the analysis of complex proteins and peptides in blood, particularly in proteomic analysis using a mass spectrometer (Anderson L. et al., Mol CellProteomics, 5: 375-88, 2006; DeSouza, L. V. et al., Anal. Chem., 81: 3462-70, 2009).

In the present invention, the expression levels of the polypeptides mentioned in the present invention may be measured by the multiple-reaction monitoring method.

In the present invention, to analyze the expression levels of the polypeptides mentioned in the present invention by the multiple-reaction monitoring method, the mass-to-charge ratio values (m/z values) of the target peptides may be used, and information about the m/z values is shown in Table 1 below, without being limited thereto.

TABLE 1

| | | | Acces- | | |
|---|---|---|---|---|---|
| | | | sion | | |
| No | Gene | Protein | No. | Sequence | M + H |
| 1 | APOC1 | Apolipoprotein | P02654 | TPDVSSALDK | 1032.5280 |
| | | C1 | | | |
| 2 | CHL1 | Neural cell | O00533 | VIAVNEVGR | 966.5524 |
| | | adhesion | | | |
| | | molecule | | | |
| | | L1-like | | | |
| 3 | MMP9 | Matrix metal- | P14780 | AVIDDAFAR | 977.5051 |
| | | loproteinase-9 | | | |
| 4 | PRDX6 | Peroxiredoxin- | P30041 | LSILYPATTGR | 1191.6732 |
| | | 6 | | | |
| 5 | PRG4 | Proteoglycan 4 | Q92954 | AIGPSQTHTIR | 1180.6433 |
| 6 | PPBP | Platelet basic | P02775 | TTSGIHPK | 840.4574 |
| | | protein | | | |
| 7 | FN1 | Fibronectin | P02751 | STTPDITGYR | 1110.5426 |
| 8 | VWF | von Willebrand | P04275 | ILAGPAGDSNV | 1240.6896 |
| | | factor | | VK | |
| 9 | CLU | Clusterin | P10909 | TLLSNLEEAK | 1117.6099 |

In the present invention, the polypeptide may be APOC1 (apolipoprotein C1), CHL1 (neural cell adhesion molecule L1-like), MMP9 (matrix metalloproteinase-9), PRDX6 (peroxiredoxin-6), PRG4 (proteoglycan 4), PPBP (platelet basic protein), FN1 (fibronectin), VWF (von Willebrand factor), or CLU (clusterin). The expression level of each polypeptide present in a biological sample isolated from a subject of interest may be measured using the target peptide of each of the polypeptides.

In the present invention, as an internal standard substance, any internal standard substance that is generally used in the multiple-reaction monitoring analysis may be used. For example, E. coli beta-galactosidase may be used.

In addition, in the present invention, in order to measure the absolute amount of the polypeptide in blood, a specific peptide synthesized by substituting some amino acids of the target peptide with a stable isotope may be used as an internal standard substance. In this case, the amino acids substituted with the isotope may be lysine or arginine, without being limited thereto. Here, as the synthesized peptide, an isolated peptide with a purity of 95% or higher may be used.

In the present invention, the internal standard substance may comprise at least one radioisotope selected from the group consisting of ²H, ³H, ¹¹C, ¹³C, ¹⁴C, ¹³N, ¹⁵N, ¹⁵O, ¹⁷O and ¹⁸O, but is not limited thereto and may comprise any kind of isotope that may be used as a comparison group to measure the absolute amount of the polypeptide.

Meanwhile, in the present invention, an agent for measuring the expression level of the gene encoding the polypeptide may comprise at least one selected from the group consisting of a primer, a probe, and an antisense oligonucleotide, which bind specifically to the gene.

In the present invention, to measure the presence and expression level of the gene encoding the polypeptide, an analysis method of measuring the mRNA level of the gene may be used. Examples of the analysis method include, but are not limited to, reverse transcription-polymerase chain reaction (RT-PCR), competitive RT-PCR, real-time RT-PCR, RNase protection assay (RPA), Northern blotting, and DNA chip assay.

In the present invention, wherein the measured expression level of the polypeptide or the gene encoding the polypeptide in the biological sample isolated from the subject of interest is higher or lower than that in a normal control group, it may be predicted that the subject has a high likelihood of onset of the cancer.

In the present invention, it is possible to predict responsiveness to therapy by measuring the expression level of the polypeptide or the gene encoding the same in the biological sample isolated from the subject of interest.

In the present invention, it is possible to predict the prognosis of a subject of interest, preferably the prognosis after surgical operation, by measuring the expression levels of either the polypeptides represented by SEQ ID NOs: 1 to 9, or the genes encoding the polypeptides, in the biological sample isolated from the subject of interest. Here, the subject of interest may be a subject who has undergone surgical resection due to cancer. In the present invention, it is possible to predict the stage of cancer in the subject of interest by measuring the expression level of the polypeptide or the gene encoding the polypeptide in the biological sample isolated from the subject of interest.

In the present invention, the “stage” refers to the extent to which cancer cells have spread or the stage of cancer progression. The international classification according to the status of cancer progression generally follows the TNM stage classification. Here, ‘T (Tumor Size)’ is a classification according to the size of the primary tumor, ‘N (Lymph Node)’ is a classification according to the degree of lymph node metastasis, and ‘M (Metastasis)’ is a classification according to whether cancer has metastasized to other organs. Detailed classification for T, N and M is shown in Table 2 below, and the stage classification of cancer according to T, N and M is shown in Table 3 below.

TABLE 2

| TNM stage | | Definition |
|---|---|---|
| Size | T0 | Tumor where tumor cells show the appearance of |
| of the | | a malignant tumor, but are confined to the |
| primary | | mucosa or epithelium, and have not yet |
| tumor | | invaded the basement membrane. |
| (T stage) | T1 | A lesion or tumor confined to the organ of origin, |
| | | which is mobile and has not invaded adjacent |
| | | and surrounding tissues. |
| | T2 | A tumor with a size of about 2 to 5 cm. |
| | T3 | A tumor having a size larger than T2 but confined |
| | | to the organ of origin. |
| | T4 | A tumor that invaded and infiltrated |
| | | surrounding tissues. |
| Lymph | N0 | There is no evidence of lymph node involvement. |
| node | N1 | Invades one palpable and mobile lymph node |
| status | | (1 to 2 cm or larger, usually up to 3 cm in size) |
| (N stage) | | limited to the first station. |
| | N2 | Palpable, partially mobile and firm or hard |
| | | lymph nodes. There is microscopic |
| | | evidence of invasion, and involved |
| | | nodes are clinically entangled and show |
| | | contralateral or bilateral involvement (3 to 5 cm). |
| | N3 | Lymph nodes that are completely fixed, pass |
| | | through the capsule, are completely |
| | | fixed to bones, large blood vessels, skin, |
| | | nerves, etc., and have a size of 6 cm or more. |
| Distant | M0 | There is no distant metastasis. |
| metastasis | M1 | There is distant metastasis. |
| (M stage) | | |

TABLE 3

| | | | | |
|---|---|---|---|---|
| Stage | | | | |
| classification | T1 | T2 | T3 | T4 |

| | | |
|---|---|---|
| N0 | Stage 1 | Stage 2 |

| | |
|---|---|
| N1 | Stage 3 |
| N2 | |
| N3 | |
| M1 | Stage 4 |

In the present invention, it is possible to predict the likelihood of recurrence of cancer by measuring the expression level of the polypeptide or the gene encoding the polypeptide in the biological sample isolated from the subject of interest.

In the present invention, the types of cancer have already been described in detail, detailed description thereof will be omitted below to avoid excessive overlapping.

According to a specific embodiment of the present invention,
the fragment of the APOC1 polypeptide has the amino acid sequence of SEQ ID NO:1 (TPDVSSALDK);
the fragment of the CHL1 polypeptide has the amino acid sequence of SEQ ID NO:2 (VIAVNEVGR);
the fragments of the MMP9 polypeptide has the amino acid sequence of SEQ ID NO:3 (AVIDDAFAR);
the fragment of the PRDX6 polypeptide has the amino acid sequence of SEQ ID NO:4 (LSILYPATTGR);
the fragment of the PRG4 polypeptide has the amino acid sequence of SEQ ID NO:5 (AIGPSQTHTIR);
the fragment of the PPBP polypeptide has the amino acid sequence of SEQ ID NO:6 (TTSGIHPK);
the fragment of the FN1 polypeptide has the amino acid sequence of SEQ ID NO:7 (STTPDITGYR);
the fragment of the VWF polypeptide has the amino acid sequence of SEQ ID NO:8 (ILAGPAGDSNVVK); and
the fragment of the CLU polypeptide has the amino acid sequence of SEQ ID NO:9 (TLLSNLEEAK).

According to a specific embodiment of the present invention, the biological sample is whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid.

According to a specific embodiment of the present invention, the agent for measuring the expression level of the polypeptide comprises at least one selected from the group consisting of an antibody, an oligopeptide, a ligand, a peptide nucleic acid (PNA), and an aptamer, which bind specifically to the polypeptide.

According to a specific embodiment of the present invention, the measurement of the expression level of the polypeptide is performed by protein chip assay, immunoassay, ligand binding assay, MALDI-TOF (Matrix Assisted Laser Desorption/Ionization Time of Flight Mass Spectrometry) assay, SELDI-TOF (Surface Enhanced Laser Desorption/Ionization Time of Flight Mass Spectrometry) assay, radioimmunoassay, radioimmunodiffusion, Ouchterlony immunodiffusion, rocket immunoelectrophoresis, immunohistochemical staining, complement fixation assay, two-dimensional electrophoresis assay, liquid chromatography-mass spectrometry (LC-MS), LC-MS/MS (liquid chromatography-mass spectrometry/mass spectrometry), Western blotting, and ELISA (enzyme-linked immunosorbent assay).

According to a specific embodiment of the present invention, the measurement of the expression level of the polypeptide is performed by a multiple-reaction monitoring (MRM) method.

According to a specific embodiment of the present invention,
the mass-to-charge ratio (m/z) of the polypeptide represented by SEQ ID NO:1 is 1032.5280 or in the range of 1032.5280±1 when the z value is 1;
the mass-to-charge ratio (m/z) of the polypeptide represented by SEQ ID NO:2 is 966.5524 or in the range of 966.5524±1 when the z value is 1;
the mass-to-charge ratio (m/z) of the polypeptide represented by SEQ ID NO:3 is 977.5051 or in the range of 977.5051±1 when the z value is 1;
the mass-to-charge ratio (m/z) of the polypeptide represented by SEQ ID NO:4 is 1191.6732 or in the range of 1191.6732±1 when the z value is 1;
the mass-to-charge ratio (m/z) of the polypeptide represented by SEQ ID NO:5 is 1180.6433 or in the range of 1180.6433±1 when the z value is 1;
the mass-to-charge ratio (m/z) of the polypeptide represented by SEQ ID NO:6 is 840.4574 or in the range of 840.4574±1 when the z value is 1;
the mass-to-charge ratio (m/z) of the polypeptide represented by SEQ ID NO:7 is 1110.5426 or in the range of 1110.5426±1 when the z value is 1;
the mass-to-charge ratio (m/z) of the polypeptide represented by SEQ ID NO:8 is 1240.6896 or in the range of 1240.6896±1 when the z value is 1; and
the mass-to-charge ratio (m/z) of the polypeptide represented by SEQ ID NO:9 is 1117.6099 or in the range of 1117.6099±1 when the z value is 1.

According to a specific embodiment of the present invention, the multiple-reaction monitoring method is performed using, as an internal standard substance, either a synthetic peptide obtained by substituting certain amino acids of each of the polypeptides with an isotope, or E. coli beta-galactosidase.

According to a specific embodiment of the present invention, the synthetic peptide has the same sequence as the sequence represented by SEQ ID NO:1, 2, 3, 4, 5, 6, 7, 8, or 9 and contains a stable isotope.

According to a specific embodiment of the present invention, the stable isotope is a stable isotope of any one or more elements selected from the group consisting of carbon and nitrogen.

According to a specific embodiment of the present invention, the measurement of the expression level of the gene encoding the polypeptide is performed by reverse transcription-polymerase chain reaction (RT-PCR), competitive RT-PCR, real-time RT-PCR, RNase protection assay (RPA), Northern blotting, or DNA chip assay.

According to a specific embodiment of the present invention,
if the measured expression levels of the CHL1 (neural cell adhesion molecule L1-like), MMP9 (matrix metalloproteinase-9), PRG4 (proteoglycan 4), PPBP (platelet basic protein), FN1 (fibronectin), VWF (von Willebrand factor) and CLU (clusterin) polypeptides or the genes encoding the same in the biological sample isolated from the subject of interest are higher than those in a normal control group, and
if the measured expression levels of the APOC1 (apolipoprotein C1) and PRDX6 (peroxiredoxin-6) polypeptides or the genes encoding the same in the biological sample are lower than those in the normal control group, the likelihood of onset of the cancer is predicted to be high.

According to a specific embodiment of the present invention, the method of providing information predicts the responsiveness of the subject of interest to an anticancer drug.

According to another aspect of the present invention, the present invention provides a method for screening a composition for preventing or treating cancer, comprising steps of:
(a) bringing a candidate substance into contact with a biological sample containing at least one polypeptide selected from the group consisting of polypeptides represented by SEQ ID NO:1 (TPDVSSALDK), SEQ ID NO:2 (VIAVNEVGR), SEQ ID NO:3 (AVIDDAFAR), SEQ ID NO:4 (LSILYPATTGR), SEQ ID NO:5 (AIGPSQTHTIR), SEQ ID NO:6 (TTSGIHPK), SEQ ID NO:7 (STTPDITGYR), SEQ ID NO:8 (ILAGPAGDSNVVK), and SEQ ID NO:9 (TLLSNLEEAK), respectively, or a gene encoding the polypeptide, or cells expressing the polypeptide or gene; and
(b) measuring the expression level of at least one polypeptide selected from the group consisting of polypeptides represented by SEQ ID NO:1 (TPDVSSALDK), SEQ ID NO:2 (VIAVNEVGR), SEQ ID NO:3 (AVIDDAFAR), SEQ ID NO:4 (LSILYPATTGR), SEQ ID NO:5 (AIGPSQTHTIR), SEQ ID NO:6 (TTSGIHPK), SEQ ID NO:7 (STTPDITGYR), SEQ ID NO:8 (ILAGPAGDSNVVK), and SEQ ID NO:9 (TLLSNLEEAK), respectively, or the gene encoding the polypeptide, in the biological sample,
wherein, the measured expression level of the polypeptide of SEQ ID NO:2, SEQ ID NO:3, SEQ ID NO:5, SEQ ID NO:6, SEQ ID NO:7, SEQ ID NO:8 or SEQ ID NO:9 or the gene encoding the same in the biological sample isolated from the subject of interest decreased compared to that in a normal control group and;
the measured expression level of the polypeptide of SEQ ID NO:1 or SEQ ID NO:4 or the gene encoding the same increased compared to that in the normal control group, the candidate substance is determined as the composition for preventing or treating cancer.

In the present invention, the sample and the cancer have already been described in detail, and thus detailed description thereof will be omitted to avoid excessive overlapping.

In the present invention, the biological sample may be allowed to react with the candidate drug for preventing or treating cancer in a manipulated or unmanipulated state.

The term “candidate substance” in the present invention refers to an unknown substance that is added to a sample containing cells expressing the genes of the present invention and is used in screening to examine whether or not it affects the activities or expression levels of these genes. Examples of the test substance include, but are not limited to, compounds, nucleotides, peptides, and natural extracts. The step of measuring the expression level or activity of the gene in the biological sample treated with the test substance may be performed by various expression level and activity measurement methods known in the art.

According to another aspect of the present invention, the present invention provides a system comprising:
an input unit configured to receive an input value;
a reading unit comprising a machine learning model pre-trained to read whether breast cancer has occurred; and
an output unit configured to output whether breast cancer has occurred,
wherein the input value is a measured value for the expression level of at least one polypeptide selected from the group consisting of SEQ ID NO:1 (TPDVSSALDK), SEQ ID NO:2 (VIAVNEVGR), SEQ ID NO:3 (AVIDDAFAR), SEQ ID NO:4 (LSILYPATTGR), SEQ ID NO:5 (AIGPSQTHTIR), SEQ ID NO:6 (TTSGIHPK), SEQ ID NO:7 (STTPDITGYR), SEQ ID NO:8 (ILAGPAGDSNVVK) and SEQ ID NO:9 (TLLSNLEEAK), in a biological sample.

The term “machine learning” in the present invention refers to algorithms and statistical models that computer systems use to perform tasks without explicit instructions, relying on patterns and inferences. The machine learning model that is used in the present invention may be specifically a deep learning, logistic regression, support vector machine (SVM), random forest, or gradient boosting algorithm GBM) model, more specifically a deep learning model, but is not limited thereto and may be any type of machine learning model that can diagnose breast cancer using the data obtained by measuring the expression levels of the biomarkers of the present invention.

According to a specific embodiment of the present invention, the machine learning model is a deep learning model.

According to a specific embodiment of the present invention, the biological sample is blood.

In the present invention, the input value of the machine learning model may be a measured value for the expression level of the polypeptide in “blood,” a biological sample, without being limited thereto. Here, the “biological sample” refers to any material, biological fluid, tissue or cells obtained or derived from the subject. For example, the biological sample may include whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid. Preferably, the biological sample may be a liquid biopsy (e.g., patient's tissue, cells, blood, serum, plasma, saliva, sputum or ascites, etc.) collected for histopathological examination by inserting a hollow needle or the like into an in vivo organ without incision of the skin of a patient having a high likelihood of onset of cancer.

According to a specific embodiment of the present invention, the measured value for the expression level of the polypeptide is a quantitative value obtained by mass spectrometry.

In the present invention, mass spectrometry for measuring the expression level of the polypeptide has already been described in detail, detailed description thereof will be omitted below to avoid excessive overlapping.

According to a specific embodiment of the present invention, the mass spectrometry is liquid chromatography-tandem mass spectrometry (LC-MS/MS).

According to another aspect of the present invention, the present invention provides a method for diagnosing cancer, comprising a step of administering to a subject a composition comprising an agent for measuring the expression level of at least one polypeptide selected from the group consisting of APOC1 (apolipoprotein C1), CHL1 (neural cell adhesion molecule L1-like), MMP9 (matrix metalloproteinase-9), PRDX6 (peroxiredoxin-6), PRG4 (proteoglycan 4), PPBP (platelet basic protein), FN1 (fibronectin), VWF (von Willebrand factor), and CLU (clusterin), or a fragment thereof, or a gene encoding the polypeptide or fragment thereof.

### Advantageous Effects

The features and advantages of the present invention are summarized as follows:
(a) The present invention discovers an agent for measuring the expression level of proteins (or fragments thereof), which are specifically expressed in cancer, or genes encoding these proteins, and provides a method of diagnosing cancer using the agent.
(b) According to the present invention, specific proteins and fragments thereof useful for cancer diagnosis are discovered among blood proteins, and they may be used as diagnostic biomarkers to diagnose in particular breast cancer simply and accurately at an early stage, thereby significantly lowering the mortality rate of patients with related diseases.

### BRIEF DESCRIPTION OF DRAWINGS

FIG. 1 is a diagram related to a library generated by analyzing peptides in a blood sample. FIG. 1a shows the process of generating a library (PepQuant Library) composed of mass spectrometry data for peptides present in a blood sample, and shows a description of the sample, protein pretreatment, and the mass spectrometry process. FIG. 1b shows the process of identifying quantifiable target peptides by comparing chromatograms produced by a triple quadrupole (MS/MS) system and a chromatogram of a standard. FIG. 1c shows the distribution of quantifiable peptides, which is the log-scale distribution for 124 blood proteins whose concentrations are known in the Blood Atlas among quantifiable proteins in the PepQuant library. FIG. 1d shows the histogram distribution for the blood proteins shown in FIG. 1c. FIG. 1e shows the number of quantifiable markers for each of serum and plasma in the PepQuant library.

FIG. 2 is a diagram showing the architecture of a deep learning model for onset of a breast cancer diagnosis model.

FIG. 3 shows the results of an experiment conducted to confirm the accuracy of diagnosis by the biomarker combination and deep learning algorithm selected in the present invention. FIG. 3a shows ROC curves showing the results of analyzing the accuracy of diagnosis by the biomarker combination and deep learning algorithm selected in the present invention. FIG. 3b is a box plot showing the results of analyzing the diagnostic accuracy by the biomarker combination and deep learning algorithm selected in the present invention for breast cancer at different stages and other cancer.

### MODE FOR INVENTION

Hereinafter, the present invention will be described in more detail by way of examples. These examples are only for illustrating the present invention in more detail, and it will be apparent to those skilled in the art that the scope of the present invention according to the subject matter of the present invention is not limited by these examples.

### EXAMPLES

### Experimental Methods

### PepQuant Library Construction

An MRM library (PepQuant Library) was constructed to secure the maximum number of reproducible and quantifiable peptides within a very fast analysis time of 10 minutes by utilizing whole serum and plasma without depletion of highly abundant proteins.

For this purpose, the present inventors collected a list of 3,338 different proteins known to exist in blood from the Secretome Database (Science, 2019), Blood atlas DB, and papers. From them, 4,683 surrogate peptides (“surrogate peptide” refers to a peptide that has a sequence existing only in a specific protein and can represent a specific protein) were selected in silico using an in-house algorithm and selection logic.

The selected 4,683 selected peptides were all synthesized, and the individual synthesized peptides were used to check whether the same peptides present in the actual serum sample were detected. That is, the retention time and fragmentation pattern of the synthetic standard peptide were first determined, and if the same retention time and the same fragmentation pattern were observed in the blood sample, it was determined that this peptide was detected in the blood. The MRM library (PepQuant library) corresponds to a comprehensive LC-MSMS chromatogram database of 4,683 surrogate peptides generated from 3,338 proteins and determined to be the most stable form. This library contains both 4,683 standard peptide chromatograms and endogenous peptide chromatograms. An MRM library named ‘PepQuant Library’ was constructed using a total of 148 blood samples. These samples included 6 different cancer types (40 breast cancer, 20 pancreatic cancer, 20 thyroid cancer, 20 ovarian cancer, 18 colorectal cancer, and 30 disease-free normal blood samples).

### Sample Collection and Preprocessing

### Sample Collection

From 2019 to 2020, blood samples were collected from a total of 13 institutions through IRB to discover breast cancer and cancer diagnostic markers. Of these, 40 cancer samples and 30 normal samples collected from Seoul National University Hospital were used. In addition, for the study of cancer markers other than breast cancer and correlations, serum samples collected in 2020, that is, 20 cases of pancreatic cancer, 20 cases of thyroid cancer, 20 cases of ovarian cancer, 20 cases of lung cancer, and 18 cases of colorectal cancer, were used.

### —Sample Preprocessing Before MRMAnalysis

Serum/plasma samples were used without depletion of highly abundant proteins (albumin, transferrin, immunoglobulin, etc.). 5 μL of serum was added to a buffer containing urea and dithiothreitol (DTT) reagent. Through this, the cysteine disulfide bonds were cleaved and the protein structure was disrupted. This sample was incubated at 35° C. for 1 hour and 30 minutes. After incubation, the sample was cooled to room temperature, and iodoacetamide (IAA) was added thereto, followed by incubation at room temperature for 30 minutes in the dark. The cleaved portion of the disulfide bond was alkylated by the IAA to prevent recombination. For subsequent trypsin digestion, the sample was diluted with ammonium bicarbonate buffer, and 5 μg (about 1:50 (w/w)) of trypsin was added, followed by incubation at 37° C. for 16 hours. Thereafter, the trypsin reaction was terminated using 10% TFA, and desalting was performed. The completely dried sample was stored at −80° C. until mass spectrometry. For mass spectrometry, dried samples were resuspended in 0.10% formic acid (FA).

### — Mass Spectrometry

The mass spectrometers were Qtrap 5500 and Qtrap 5500 Plus (Sciex, USA), Analyst 1.7.2 was used as the analysis software, and Multiquant (3.0.2) was used as the quantification program. LC separation was performed using a C18 reversed-phase column, and analysis was performed in MRM mode.

### Reagents

Heavy peptide standards for each biomarker used in this development were synthesized in the GMP (Good Manufacturing Practice) facility of Bertis Inc. (Republic of Korea). The stock solution of each heavy peptide standard was stored in a deep freezer at −80° C. and used after dilution whenever necessary.

### — PepQuant Library Construction and Marker Discovery Results

An MRM library named “pepQuant-library” was constructed to select peptides that are detectable in a very short gradient time of 10 minutes without depletion of highly abundant proteins. This library contains a database of all 4,683 standard peptide chromatograms and endogenous peptide chromatograms.

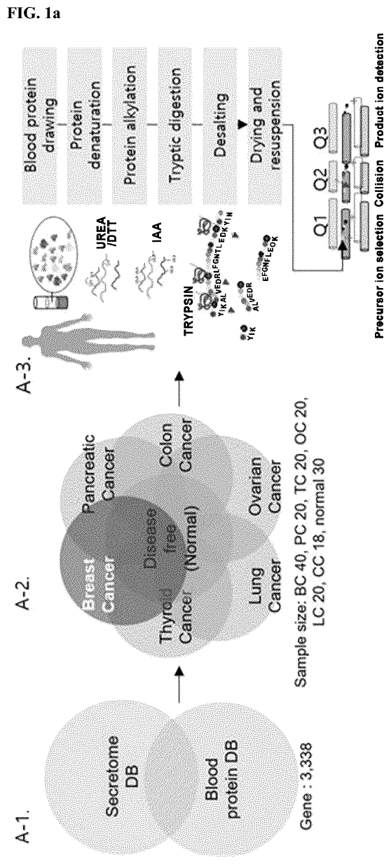

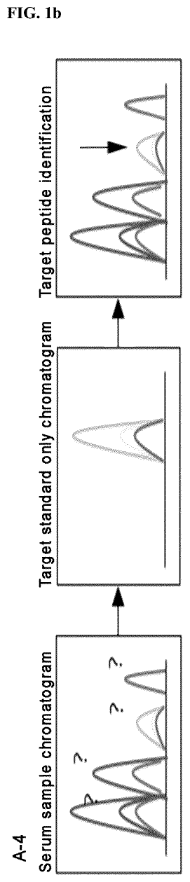

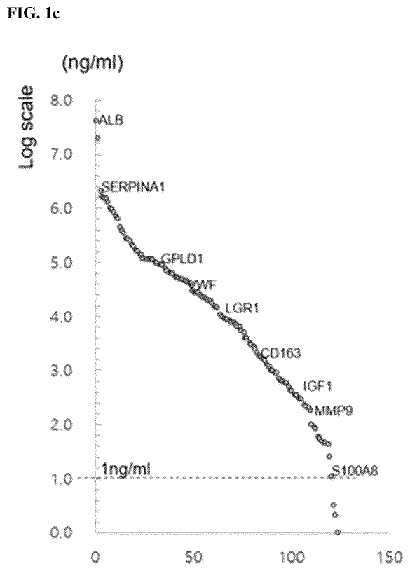

Among blood peptides whose retention time and second product pattern match those of the synthetic standard, these peptides with an S/N ratio of 3 or more were defined as detectable peptides. Conversely, if the fragmentation pattern or retention time did not match, the target peptide was considered not detected. Based on the chromatogram information of 4,683 synthetic standard peptides, 452 proteins and 852 peptides present in actual blood samples were detected. As a result of examining the distribution of 124 proteins with known concentrations among detectable peptides, it was confirmed that these proteins were present in a wide concentration range from 40 mg/ml or more to 1 ng/ml (FIG. 1). These results show that much more proteins than those in other studies under similar conditions were detected (Table 4).

TABLE 4
Comparison of the number of proteins that are measurable
without depletion of highly abundant proteins

| | | Protein | Mass | Gradient | |
|---|---|---|---|---|---|
| | Detection | range | type | time | Year |
| AJ Percy et al. | 142 | 31 mg to | LC-MSMS | 45 min | 2013 |
| | | 44 ng/ml | | | |
| PE Geyer et al. | 313 | NA | Orbitrap | 20 min | 2016 |
| A Kraut et al. | 280 | NA | Orbitrap | 60 min | 2019 |
| PepQuant-library | 452 | 40 mg to | LC-MSMS | 10 min | 2022 |
| | | 1 ng/ml | | | |

### Algorithm Development Method

### — Use of Samples

This clinical trial was conducted using serum samples collected for a prospective multicenter clinical trial of breast cancer markers (KCT 0004847). For the clinical trial, a total of 649 plasma and serum samples were collected from 13 hospitals from 2019 to 2020. However, the serum samples available in sufficient quantity for the discovery and development of additional breast cancer markers in serum samples, which was one of the goals of the clinical trial, were limited to 402 serum samples from 12 hospitals. In addition, all the samples are adult female samples. The normal serum samples used in the clinical trial belong to the BI-RADS 1 or 2 category in which cancer is not detected in breast imaging. Additionally, all samples should satisfy the condition that there has been no other cancer or recurrence within 5 years. Cancer samples belong to samples collected from breast cancer patients before biopsy. The number of samples from each hospital was as follows: Seoul National University Hospital (187 normal samples), Seoul National University Bundang Hospital (14 cancer samples), Dankook University Hospital (27 cancer samples), Chung-Ang University Hospital (26 cancer samples), Hallym University Gangnam Sacred Heart Hospital (13 cancer samples), National Cancer Center (22 cancer samples), Myongji Hospital (25 cancer samples), Hanyang University Hospital (9 cancer samples), The Catholic University of Korea, Seoul, St. Mary's Hospital (11 cancer samples), Korea University Anam Hospital (14 cancer samples), Korea University Guro Hospital (29 cancer samples), and Gyeongsang National University Hospital (25 cancer samples) (Table 5).

The people from whom the samples were collected were women over 20 years old, and people in their 40 s and 50 s accounted for 63% of all the women. The normal group corresponds to those who have not developed any cancer, including other cancers, for 5 years and do not need a breast biopsy for imaging purposes. The cancer patient group refers to cases confirmed pathologically through a breast biopsy. The distribution by stage for samples with known stage was as follows: TNM stage 0 (9.5%), stage 1 (38.1%), stage 2 (35.7%), and stage 3-4 (16.7%).

98 serum samples from five major cancers other than breast cancer (20 thyroid cancer samples, 20 ovarian cancer samples, 20 pancreatic cancer samples, 18 lung cancer sample, and 20 colorectal cancer samples) were collected in a retrospective clinical trial for discovery and validation of breast cancer markers, and the retrospective clinical trial was approved by the Institutional Review Board of Seoul National University Hospital (Approval No. H-1911-085-1079).

TABLE 5
Algorithm development set: total sample information

| | | | | Density_grade | | | | | TNM stage | | | | |
|---|---|---|---|---|---|---|---|---|---|---|---|---|---|
| | | | BMI | (1~4) | | | | | classification | | | | |
| Group | Age | Number | (AVG.) | 1 | 2 | 3 | 4 | NA | 0 | 1 | 2 | 3-4 | NA |
| Normal | 20 to | 30 | 20.8 | 0 | 0 | 4 | 6 | 20 | — | — | — | — | — |
| | 39 | | | | | | | | | | | | |
| | 40 to | 68 | 23.1 | 1 | 0 | 26 | 28 | 13 | — | — | — | — | — |
| | 49 | | | | | | | | | | | | |
| | 50 to | 62 | 23.6 | 0 | 5 | 33 | 17 | 6 | — | — | — | — | — |
| | 59 | | | | | | | | | | | | |
| | 60 to | 26 | 24.3 | 1 | 5 | 17 | 2 | 1 | — | — | — | — | — |
| | 69 | | | | | | | | | | | | |
| | 70 + | 5 | 22.1 | 0 | 2 | 3 | 0 | 0 | — | — | — | — | — |
| Subtotal | | 187 | | 2 | 12 | 81 | 52 | 40 | | | | | |
| Cancer | 20 to | 9 | 21.5 | 0 | 2 | 5 | 1 | 0 | 0 | 2 | 5 | 1 | 1 |
| patients | 39 | | | | | | | | | | | | |
| | 40 to | 66 | 23.6 | 8 | 16 | 19 | 4 | 1 | 8 | 16 | 19 | 5 | 18 |
| | 49 | | | | | | | | | | | | |
| | 50 to | 85 | 24.0 | 3 | 29 | 18 | 11 | 2 | 3 | 29 | 18 | 14 | 21 |
| | 59 | | | | | | | | | | | | |
| | 60 to | 73 | 24.9 | 7 | 22 | 21 | 5 | 0 | 7 | 22 | 21 | 6 | 17 |
| | 69 | | | | | | | | | | | | |
| | 70 + | 28 | 25.1 | 2 | 10 | 10 | 2 | 0 | 2 | 10 | 10 | 2 | 4 |

| | | | | | | | | | | | |
|---|---|---|---|---|---|---|---|---|---|---|---|
| Subtotal | 215 | 3 | 14 | 24 | 7 | 213 | 20 | 80 | 75 | 35 | 61 |
| Total | 402 | 22 | 91 | 155 | 75 | 43 | 20 | 80 | 75 | 35 | 61 |

### — Blood Separation, Sample Preprocessing Before MRM Analysis, and Mass Spectrometry

The same methods as the blood separation, sample preprocessing before MRM analysis, and mass spectrometry methods used in construction of the PepQuant library of the present invention were used.

### — Analysis Markers

The final markers discovered by the PepQuant-library were used.

### — Algorithm Model Development

70% of normal samples and 70% of cancer samples were randomly classified and used to develop an algorithm model. Random sampling was carried out a total of 5 times, and validation (test) is performed using the remaining 30% of the set used for development (Table 6). To develop a breast cancer diagnosis model, several algorithms were tested. Deep learning, Logistic regression, Random forest, and Gradient boosting algorithms were used, and Python models were used for all the algorithms. Logistic regression and Random forest algorithms were trained using basic parameters in the scikit-learn framework, and for the gradient boosting algorithm, the basic parameters of the XGboost framework were used. The deep learning model was developed using the torch framework.

TABLE 6
Input data information for selecting
the optimal algorithm model

| | Algorithm | Test (30%) | |
|---|---|---|---|
| | development | external | Total |
| | (70%) | validation | (100%) |
| Normal | 131 | 56 | 187 |
| Age 20-39 | 20 | 8 | |
| Age 40-59 | 92 | 36 | |
| Age 60-79 | 19 | 11 | |
| Age 80+ | 0 | 0 | |
| Unknown | 0 | 1 | |
| Breast cancer | 150 | 65 | 215 |
| Age 20-39 | 3 | 3 | |
| Age 40-59 | 82 | 35 | |
| Age 60-79 | 51 | 19 | |
| Age 80+ | 4 | 1 | |
| Unknown | 10 | 7 | |
| Stage 0 | 14 | 2 | |
| Stage 1 | 33 | 22 | |
| Stage 2 | 41 | 16 | |
| Stage 3+ | 10 | 3 | |
| unknown | 52 | 22 | |
| Total | 281 | 121 | 402 |

Among the four deep learning/machine learning models, the deep learning model was found to have the highest AUC value. Therefore, the deep learning model that showed the highest AUC value was selected as the final algorithm model.

### — Deep Learning Model Architecture

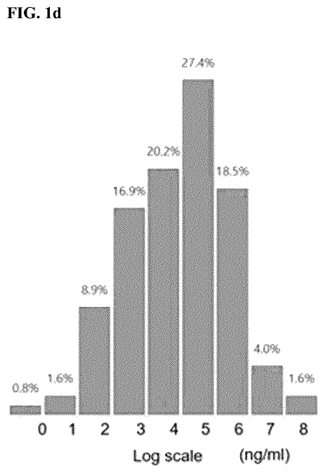

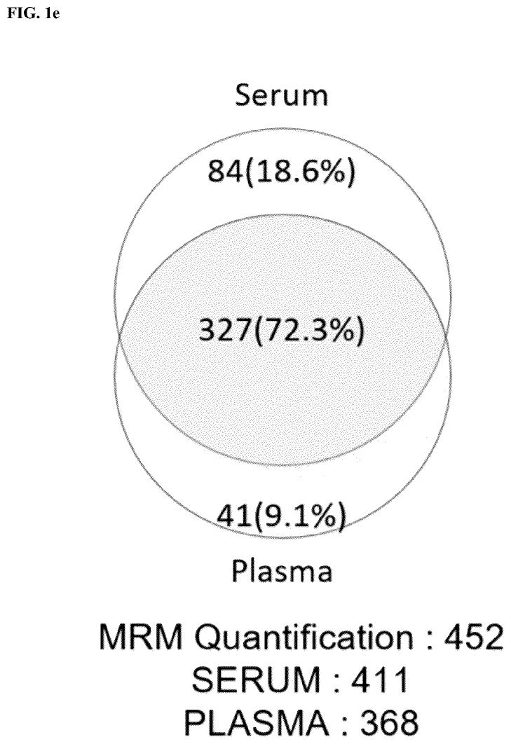

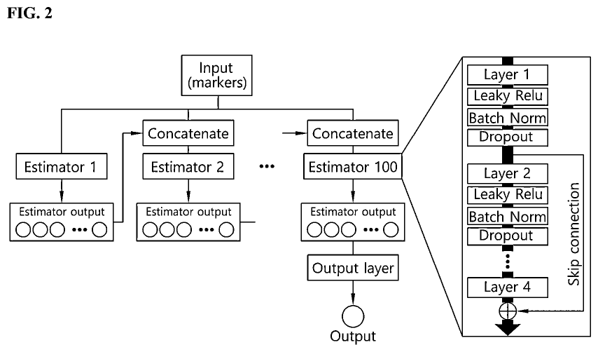

The structure of the MastoCheck2 model is a deep learning model designed based on the architecture of GrowNet, a type of deep learning algorithm. For reference, GrowNet is a model structured as a neural network that configures the structure of the Gradient Boosting Machine (GBM), also known as the Gradient Boosting algorithm. GBM combines multiple weak learners sequentially to form a strong learner. Consequently, this algorithm model can be repeatedly composed of several weak learners. The final output is expressed as a probability value between 0 and 1, indicating the likelihood of a positive diagnosis. (FIG. 2).

### — Model Validation

A total of 500 samples, including other cancer samples, were used for model training and validation. These samples comprised 187 non-cancer samples that were radiologically unlikely to be breast cancer (BI-RADS C1, C2) and had no experience of onset of cancer within 5 years, 98 samples from patients with cancer other than breast cancer, and 215 preoperative samples from confirmed breast cancer patients (Table 7). 70% of normal samples and 70% of cancer samples were randomly classified and used to develop an algorithm model, and the remaining 30% were used for model validation.

TABLE 7
Input data information used to develop deep learning algorithm

| | Sample | Training | Test | Total |
|---|---|---|---|---|
| | category | set (70%) | set (30%) | (100%) |
| Healthy controls | Age Total | 131 | 56 | 187 |
| | Age 20-39 | 20 | 8 | |
| | Age 40-59 | 92 | 36 | |
| | Age 60-79 | 19 | 11 | |
| | Age 80+ | 0 | 0 | |
| | Unknown | 0 | 1 | |
| Breast cancer | Age Total | 150 | 65 | 215 |
| | Age 20-39 | 3 | 3 | |
| | Age 40-59 | 82 | 35 | |
| | Age 60-79 | 51 | 19 | |
| | Age 80+ | 4 | 1 | |
| | Unknown | 10 | 7 | |
| | Stage Total | 150 | 65 | 215 |
| | Stage 0 | 14 | 2 | |
| | Stage 1 | 33 | 22 | |
| | Stage 2 | 41 | 16 | |
| | Stage 3+ | 10 | 3 | |
| | Unknown | 52 | 22 | |
| Other cancers | Other cancers total | 69 | 29 | 98 |
| | Ovarian | 14 | 6 | |
| | Pancreas | 14 | 6 | |
| | Thyroid | 14 | 6 | |
| | Colon | 14 | 6 | |
| | Lung | 13 | 5 | |
| | Total | 350 | 150 | 500 |

### —Selection of Breast Cancer Markers

Among the samples used in the library construction, 50 breast cancer samples and 50 normal samples were used. MRM analysis was performed using LC-MS/MS on 418 peptides, which are detectable in serum, within the target peptide list in the PepQuant library. Here, a protein whose abundance is significantly different between breast cancer and normal was selected as a marker. For quantification, synthetic isotope standards for each marker were used as internal standards (IS). The abundance of the analyte (A) was expressed as (A/IS ratio X IS quantity=quantification value). A total of 418 internal standards were synthesized (Bertis, Korea), and the overall sample preprocessing method was the same as the method used when constructing the PepQuant library.

The primary candidate marker selection criterion was to select markers with an average ratio of breast cancer samples to normal samples of 1.2-fold or more or 0.8-fold or less, and 30 candidate markers were selected (Table 8). The p-value was set at less than 5% using a two-sided t-test, and the Wilcoxon rank sum test was used to calculate the P-value.

TABLE 8
30 candidate markers

| | | Acces- | | |
|---|---|---|---|---|
| | | sion | | |
| No. | Gene | No. | Protein name | Sequence |
| 1 | FN1 | P02751 | Fibronectin | STTPDITGYR |
| 2 | FN1 | P02751 | Fibronectin | VDVIPVNLPGEHGQR |
| 3 | VWF | P04275 | von Willebrand | ILAGPAGDSNVVK |
| | | | factor | |
| 4 | VWF | P04275 | von Willebrand | VTVFPIGIGDR |
| | | | factor | |
| 5 | MMP9 | P14780 | Matrix metallo- | AVIDDAFAR |
| | | | proteinase 9 | |
| 6 | PRG4 | Q92954 | Proteoglycan 4 | AIGPSQTHTIR |
| 7 | PRG4 | Q92954 | Proteoglycan 4 | DQYYNIDVPSR |
| 8 | THBS1 | P07996 | Thrombospondin 1 | LVPNPDQK |
| 9 | APOC1 | P02654 | Apolipoprotein C1 | TPDVSSALDK |
| 10 | CHL1 | O00533 | Neural cell | VIAVNEVGR |
| | | | adhesion molecule | |
| | | | L1 like | |
| 11 | B2M | P61769 | Beta-2- | IQVYSR |
| | | | microglobulin | |
| 12 | LYZ | P61626 | Lysozyme C | STDYGIFQINSR |
| 13 | CTSD | P07339 | Cathepsin D | VSTLPAITLK |
| 14 | PPBP | P02775 | Platelet basic | TTSGIHPK |
| | | | protein | |
| 15 | C4BPA | P04003 | C4b-binding | LSLEIEQLELQR |
| | | | protein alpha | |
| | | | chain | |
| 16 | HBD | P02042 | Hemoglobin sub- | TAVNALWGK |
| | | | unit delta | |
| 17 | Igals3bp | Q08380 | Galectin-3- | YSSDYFQAPSDYR |
| | | | binding protein | |
| 18 | MASP1 | P48740 | Mannan-binding | SDFSNEER |
| | | | lectin serine | |
| | | | protease 1 | |
| 19 | APOF | Q13790 | Apolipoprotein F | SGVQQLIQYYQDQK |
| 20 | CPB2 | Q96IY4 | Carboxypeptidase | YSFTIELR |
| | | | B2 | |
| 21 | VCAM1 | P19320 | Vascular cell | SIDGAYTIR |
| | | | adhesion | |
| | | | protein 1 | |
| 22 | GPLD1 | P80108 | Phosphati- | GAVYVYFGSK |
| | | | dylinositol- | |
| | | | glycan-specific | |
| | | | phospholipase D | |
| 23 | FCGBP | Q9Y6R7 | IgGFc-binding | GNPAVSYVR |
| | | | protein | |
| 24 | LTF | P02788 | lactotransferrin | YYGYTGAFR |
| 25 | FCN2 | Q15485 | Ficolin-2 | VDGSVDFYR |
| 26 | PRDX6 | P30041 | Peroxiredoxin-6 | LSILYPATTGR |
| 27 | IGF1 | P05019 | Insulin-like | GFYFNKPTGYGSSSR |
| | | | growth factor1 | |
| 28 | CLU | P10909 | Clusterin | TLLSNLEEAK |
| 29 | CHGA | P10645 | Chromogranin-A | ILSILR |
| 30 | PIGR | P01833 | Polymeric | VYTVDLGR |
| | | | immunoglobulin | |
| | | | receptor | |

Thereafter, 30 candidate markers were compared and validated in 96 cancer samples and 95 normal samples, and markers with an average ratio of breast cancer samples to normal samples of 1.2-fold or more or 0.8-fold or less were used (p-value<0.05). In this process, a total of 16 markers were selected, and analytical performance evaluation for each marker was performed on a mass spectrometer, thus finally selecting 9 markers (Table 9). For reference, the analytical performance evaluation is a validation test to prove the reliability of quantitative results of breast cancer candidate markers on a mass spectrometer. The quantitative value of the corresponding peptide was measured by LC-MS/MS, and the markers were evaluated in terms of the following items: specificity (selectivity), linearity, intra-day precision/inter-day precision, stability, and media effect.

TABLE 9
List of final 9 markers

| | | | | Accession |
|---|---|---|---|---|
| No. | Gene | Protein | Sequence | No. |
| 1 | APOC1 | Apolipoprotein C1 | TPDVSSALDK | P02654 |
| 2 | CHL1 | Neural cell | VIAVNEVGR | O00533 |
| | | adhesion | | |
| | | molecule L1 like | | |
| 3 | MMP9 | Matrix metallo- | AVIDDAFAR | P14780 |
| | | proteinase-9 | | |
| 4 | PRDX6 | Peroxiredoxin-6 | LSILYPATTGR | P30041 |
| 5 | PRG4 | Proteoglycan 4 | AIGPSQTHTIR | Q92954 |
| 6 | PPBP | Platelet basic | TTSGIHPK | P02775 |
| | | protein | | |
| 7 | FN1 | Fibronectin | STTPDITGYR | P02751 |
| 8 | VWF | von Willebrand | ILAGPAGDSNVVK | P04275 |
| | | factor | | |
| 9 | CLU | Clusterin | TLLSNLEEAK | P10909 |

### Results of Algorithm Development

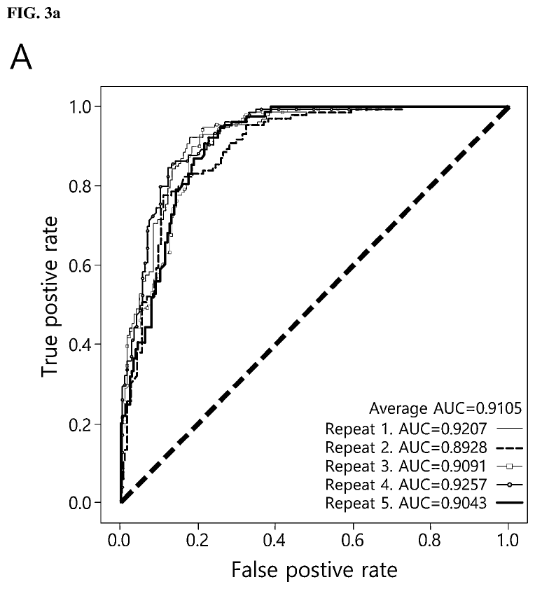

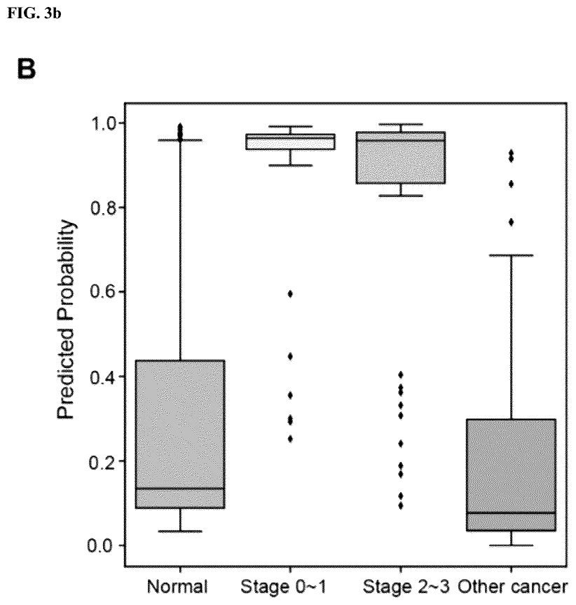

When the deep learning algorithm developed using 350 samples (70%) out of 500 samples was validated on the remaining 150 samples, an AUC value of up to 0.9207 was obtained, the average value of the learning and verification results obtained through five random assignments was also shown to be 0.9105 (FIG. 3a). In addition, it was shown that the algorithm value for stages 0 to 1 breast cancer was not lower than the value for stages 2 to 3, indicating that stage 0 to 1 breast cancer could also be diagnosed with high accuracy (FIG. 3b). In addition, other cancer samples were determined to be normal, not breast cancer, indicating that the algorithm can be more specific to breast cancer.

### Conclusion

Until now, the early diagnosis system for breast cancer has been a system relying on imaging, and thus there has been a problem in that diagnosis accuracy may be reduced due to breast density, technician skill, and old equipment. In addition, factors that reduce accessibility, such as radiation risk, discomfort, and pain, are also considered problems with the conventional early diagnosis method for breast cancer.

Therefore, high-precision blood testing can be an alternative to solving the fundamental problems of imaging diagnosis. The existing CA15-3 immunoassay, a breast cancer blood test, has the disadvantage of low accuracy for the early stage of the disease. Therefore, in order to increase accuracy, it was necessary to use a multi-marker combination.

The present inventors have demonstrated that 452 blood proteins can be quantified in LC-MS/MS within a short analysis time of 10 minutes without depletion of highly abundant proteins. Among these quantifiable markers, the final 9 markers that passed the analytical performance evaluation were selected as breast cancer screening markers. In addition, as a result of onset of and validating the algorithm formula, it was confirmed that the algorithm formula showed a high precision corresponding to a high AUC value of 0.9 or more, indicating that it can be directly applied to clinical practice. Furthermore, it was confirmed that the PepQuant library can be applied to select not only breast cancer markers but also other types of cancer and other disease markers.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

1.-26. (canceled)

27. A composition for diagnosing cancer, comprising an agent for measuring an expression level of at least one polypeptide selected from the group consisting of APOC1 (apolipoprotein C1), CHL1 (neural cell adhesion molecule L1-like), MMP9 (matrix metalloproteinase-9), PRDX6 (peroxiredoxin-6), PRG4 (proteoglycan 4), PPBP (platelet basic protein), FN1 (fibronectin), VWF (von Willebrand factor), and CLU (clusterin) or a fragment thereof, or a gene encoding the polypeptide or fragment thereof.

28. The composition of claim 27, wherein
the fragment of the APOC1 polypeptide has the amino acid sequence of SEQ ID NO:1 (TPDVSSALDK);
the fragment of the CHL1 polypeptide has the amino acid sequence of SEQ ID NO:2 (VIAVNEVGR);
the fragments of the MMP9 polypeptide has the amino acid sequence of SEQ ID NO:3 (AVIDDAFAR);
the fragment of the PRDX6 polypeptide has the amino acid sequence of SEQ ID NO:4 (LSILYPATTGR);
the fragment of the PRG4 polypeptide has the amino acid sequence of SEQ ID NO:5 (AIGPSQTHTIR);
the fragment of the PPBP polypeptide has the amino acid sequence of SEQ ID NO:6 (TTSGIHPK);
the fragment of the FN1 polypeptide has the amino acid sequence of SEQ ID NO:7 (STTPDITGYR);
the fragment of the VWF polypeptide has the amino acid sequence of SEQ ID NO:8 (ILAGPAGDSNVVK); and
the fragment of the CLU polypeptide has the amino acid sequence of SEQ ID NO:9 (TLLSNLEEAK).

29. The composition of claim 27, wherein the cancer is breast cancer.

30. The composition of claim 27, wherein the agent for measuring the expression level of the polypeptide comprises at least one selected from the group consisting of an antibody, an oligopeptide, a ligand, a peptide nucleic acid (PNA), and an aptamer, which bind specifically to the polypeptide or fragment thereof.

31. The composition of claim 30, wherein the agent for measuring the expression level of the gene encoding the polypeptide or fragment thereof comprises at least one selected from the group consisting of a primer, a probe, and an antisense oligonucleotide, which bind specifically to the gene.

32. A diagnostic kit comprising the composition of claim 1.

33. The diagnostic kit of claim 32, wherein the kit is an RT-PCR kit, a DNA chip kit, an ELISA kit, a protein chip kit, a rapid kit or a multiple-reaction monitoring (MRM) kit.

34. A method for screening a composition for preventing or treating cancer, comprising steps of:
(a) bringing a candidate substance into contact with a biological sample containing at least one polypeptide selected from the group consisting of polypeptides represented by SEQ ID NO:1 (TPDVSSALDK), SEQ ID NO:2 (VIAVNEVGR), SEQ ID NO:3 (AVIDDAFAR), SEQ ID NO:4 (LSILYPATTGR), SEQ ID NO:5 (AIGPSQTHTIR), SEQ ID NO:6 (TTSGIHPK), SEQ ID NO:7 (STTPDITGYR), SEQ ID NO:8 (ILAGPAGDSNVVK), and SEQ ID NO:9 (TLLSNLEEAK), respectively, or a gene encoding the polypeptide, or cells expressing the polypeptide or gene; and
(b) measuring an expression level of at least one polypeptide selected from the group consisting of polypeptides represented by SEQ ID NO:1 (TPDVSSALDK), SEQ ID NO:2 (VIAVNEVGR), SEQ ID NO:3 (AVIDDAFAR), SEQ ID NO:4 (LSILYPATTGR), SEQ ID NO:5 (AIGPSQTHTIR), SEQ ID NO:6 (TTSGIHPK), SEQ ID NO:7 (STTPDITGYR), SEQ ID NO:8 (ILAGPAGDSNVVK), and SEQ ID NO:9 (TLLSNLEEAK), respectively, or the gene encoding the polypeptide, in the biological sample,
wherein, the measured expression level of the polypeptide of SEQ ID NO:2, SEQ ID NO:3, SEQ ID NO:5, SEQ ID NO:6, SEQ ID NO:7, SEQ ID NO:8 or SEQ ID NO:9 or the gene encoding the polypeptide in the biological sample isolated from the subject of interest decreased compared to that in a normal control group and;
the measured expression level of the polypeptide of SEQ ID NO:1 or SEQ ID NO:4 or the gene encoding the polypeptide increased compared to that in the normal control group,
the candidate substance is determined as the composition for preventing or treating cancer.

35. A system for diagnosing breast cancer, comprising:
an input unit configured to receive an input value;
a reading unit comprising a machine learning model pre-trained to read whether breast cancer has occurred; and
an output unit configured to output whether breast cancer has occurred,
wherein the input value is a measured value for an expression level of at least one polypeptide selected from the group consisting of SEQ ID NO:1 (TPDVSSALDK), SEQ ID NO:2 (VIAVNEVGR), SEQ ID NO:3 (AVIDDAFAR), SEQ ID NO:4 (LSILYPATTGR), SEQ ID NO:5 (AIGPSQTHTIR), SEQ ID NO:6 (TTSGIHPK), SEQ ID NO:7 (STTPDITGYR), SEQ ID NO:8 (ILAGPAGDSNVVK), and SEQ ID NO:9 (TLLSNLEEAK), in a biological sample.

36. The system of claim 35, wherein the machine learning model is a deep learning model.

37. The system of claim 35, wherein the biological sample is whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid.

38. The system of claim 35, wherein the measured value for the expression level of the polypeptide is a quantitative value obtained by mass spectrometry.

39. The system of claim 35, wherein the measuring of the expression level of the polypeptide is performed by protein chip assay, immunoassay, ligand binding assay, MALDI-TOF (Matrix Assisted Laser Desorption/Ionization Time of Flight Mass Spectrometry) assay, SELDI-TOF (Surface Enhanced Laser Desorption/Ionization Time of Flight Mass Spectrometry) assay, radioimmunoassay, radioimmunodiffusion, Ouchterlony immunodiffusion, rocket immunoelectrophoresis, immunohistochemical staining, complement fixation assay, two-dimensional electrophoresis assay, liquid chromatography-mass spectrometry (LC-MS), LC-MS/MS (liquid chromatography-mass spectrometry/mass spectrometry), Western blotting, or ELISA (enzyme-linked immunosorbent assay).

40. The system of claim 35, wherein the measuring of the expression level of the polypeptide is performed by a multiple-reaction monitoring (MRM) method.

41. The system of claim 35, wherein
a mass-to-charge ratio (m/z) of the polypeptide represented by SEQ ID NO:1 is 1032.5280 or in the range of 1032.5280±1 when the z value is 1;
a mass-to-charge ratio (m/z) of the polypeptide represented by SEQ ID NO:2 is 966.5524 or in the range of 966.5524±1 when the z value is 1;
a mass-to-charge ratio (m/z) of the polypeptide represented by SEQ ID NO:3 is 977.5051 or in the range of 977.5051±1 when the z value is 1;
a mass-to-charge ratio (m/z) of the polypeptide represented by SEQ ID NO:4 is 1191.6732 or in the range of 1191.6732±1 when the z value is 1;
a mass-to-charge ratio (m/z) of the polypeptide represented by SEQ ID NO:5 is 1180.6433 or in the range of 1180.6433±1 when the z value is 1;
a mass-to-charge ratio (m/z) of the polypeptide represented by SEQ ID NO:6 is 840.4574 or in the range of 840.4574±1 when the z value is 1;
a mass-to-charge ratio (m/z) of the polypeptide represented by SEQ ID NO:7 is 1110.5426 or in the range of 1110.5426±1 when the z value is 1;
a mass-to-charge ratio (m/z) of the polypeptide represented by SEQ ID NO:8 is 1240.6896 or in the range of 1240.6896±1 when the z value is 1; and
a mass-to-charge ratio (m/z) of the polypeptide represented by SEQ ID NO:9 is 1117.6099 or in the range of 1117.6099±1 when the z value is 1.

42. The system of claim 40, wherein the multiple-reaction monitoring method uses a synthetic peptide obtained by substituting certain amino acids of each of the polypeptides with an isotope or E. coli beta-galactosidase as an internal standard substance.

43. The system of claim 42, wherein the synthetic peptide has the same sequence as the sequence represented by SEQ ID NO:1, 2, 3, 4, 5, 6, 7, 8, or 9 and comprise a stable isotope.

44. The system of claim 35, wherein the measuring of the expression level of the gene encoding the polypeptide is performed by reverse transcription-polymerase chain reaction (RT-PCR), competitive RT-PCR, real-time RT-PCR, RNase protection assay (RPA), Northern blotting, or DNA chip assay.

45. The system of claim 35,
wherein the measured expression level of the CHL1 (neural cell adhesion molecule L1-like), MMP9 (matrix metalloproteinase-9), PRG4 (proteoglycan 4), PPBP (platelet basic protein), FN1 (fibronectin), VWF (von Willebrand factor) or CLU (clusterin) polypeptide or the gene encoding the polypeptide in the biological sample isolated from the subject of interest is higher than that in a normal control group and;
the measured expression level of the APOC1 (apolipoprotein C1) or PRDX6 (peroxiredoxin-6) polypeptide or the gene encoding the polypeptide in the biological sample is lower than that in the normal control group,
a likelihood of onset of the cancer is highly predicted.

46. The system of claim 35, which predicts responsiveness of the subject of interest to an anticancer drug.


SEQUENCE LISTING (30 sequences)

SEQ ID NO: 1 - protein, 10 aa, synthetic construct
TPDVSSALDK

SEQ ID NO: 2 - protein, 9 aa, synthetic construct
VIAVNEVGR

SEQ ID NO: 3 - protein, 9 aa, synthetic construct
AVIDDAFAR

SEQ ID NO: 4 - protein, 11 aa, synthetic construct
LSILYPATTGR

SEQ ID NO: 5 - protein, 11 aa, synthetic construct
AIGPSQTHTIR

SEQ ID NO: 6 - protein, 8 aa, synthetic construct
TTSGIHPK

SEQ ID NO: 7 - protein, 10 aa, synthetic construct
STTPDITGYR

SEQ ID NO: 8 - protein, 13 aa, synthetic construct
ILAGPAGDSNVVK

SEQ ID NO: 9 - protein, 10 aa, synthetic construct
TLLSNLEEAK

SEQ ID NO: 10 - protein, 15 aa, synthetic construct
VDVIPVNLPGEHGQR

SEQ ID NO: 11 - protein, 11 aa, synthetic construct
VTVFPIGIGDR

SEQ ID NO: 12 - protein, 11 aa, synthetic construct
DQYYNIDVPSR

SEQ ID NO: 13 - protein, 8 aa, synthetic construct
LVPNPDQK

SEQ ID NO: 14 - protein, 6 aa, synthetic construct
IQVYSR

SEQ ID NO: 15 - protein, 12 aa, synthetic construct
STDYGIFQINSR

SEQ ID NO: 16 - protein, 10 aa, synthetic construct
VSTLPAITLK

SEQ ID NO: 17 - protein, 12 aa, synthetic construct
LSLEIEQLELQR

SEQ ID NO: 18 - protein, 9 aa, synthetic construct
TAVNALWGK

SEQ ID NO: 19 - protein, 13 aa, synthetic construct
YSSDYFQAPSDYR

SEQ ID NO: 20 - protein, 8 aa, synthetic construct
SDFSNEER

SEQ ID NO: 21 - protein, 14 aa, synthetic construct
SGVQQLIQYYQDQK

SEQ ID NO: 22 - protein, 8 aa, synthetic construct
YSFTIELR

SEQ ID NO: 23 - protein, 9 aa, synthetic construct
SIDGAYTIR

SEQ ID NO: 24 - protein, 10 aa, synthetic construct
GAVYVYFGSK

SEQ ID NO: 25 - protein, 9 aa, synthetic construct
GNPAVSYVR

SEQ ID NO: 26 - protein, 9 aa, synthetic construct
YYGYTGAFR

SEQ ID NO: 27 - protein, 9 aa, synthetic construct
VDGSVDFYR

SEQ ID NO: 28 - protein, 15 aa, synthetic construct
GFYFNKPTGYGSSSR

SEQ ID NO: 29 - protein, 6 aa, synthetic construct
ILSILR

SEQ ID NO: 30 - protein, 8 aa, synthetic construct
VYTVDLGR